(19) United States
(12) Patent Application Publication
(10) Pub. No.: US 2026/0094273 A1
(43) Pub. Date: Apr. 2, 2026

(54) RETINAL IMAGE DATA CORRECTION FOR MULTI- AND HYPER-SPECTRAL CUBES

(71) Applicant: OPTINA DIAGNOSTICS, INC., Montreal (CA)

(72) Inventors: Jean-Philippe SYLVESTRE, Montreal (CA); Claudia CHEVREFILS, Montreal (CA); Antoine DUROCHER-JEAN, Montreal (CA); Samin SABOKROHIYEH, Montreal (CA); Gabriel DIGNARD, Montreal (CA)

(21) Appl. No.: 19/348,154

(22) Filed: Oct. 2, 2025

Related U.S. Application Data
(63) Continuation of application No. PCT/IB2024/053209, filed on Apr. 3, 2024.
(60) Provisional application No. 63/493,819, filed on Apr. 3, 2023.

(51) Int. Cl. G06T 7/00; A61B 3/00; A61B 3/12; G06T 5/20; G06T 5/50; G06T 5/70; G06T 5/80; G06T 7/60; G06T 7/73

(52) U.S. Cl. CPC G06T 7/0014; A61B 3/0025; A61B 3/1225; G06T 5/20; G06T 5/50; G06T 5/70; G06T 5/80; G06T 7/60; G06T 7/74; A61B 2560/0233; G06T 2207/10152; G06T 2207/20221; G06T 2207/20224; G06T 2207/30041; G06T 2207/30201

(57) ABSTRACT

Previous work on normalization/calibration methods for fundus imaging captured by multispectral or hyperspectral retinal cameras may be insufficient to accurately correct retinal images, for example to identify subtle spatial-spectral features indicative of a disease. In particular, previous techniques may not account for several important factors that affect the accuracy of a captured cube of image data. Accordingly, techniques described herein include improved methods for multispectral or hyperspectral image data correction.

### CROSS-REFERENCE

The present application is a continuation of PCT Application No. PCT/IB2024/053209 filed Apr. 3, 2024, and entitled “RETINAL IMAGE DATA CORRECTION FOR MULTI-AND HYPER-SPECTRAL CUBES”, which claims priority from United States Provisional Patent Application Ser. No. 63/493,819 filed on Apr. 3, 2023, the entirety of which is incorporated by reference herein in jurisdictions allowing such incorporation by reference.

### BACKGROUND

The retina is a thin layer of tissue located at the back of the eye that is part of the fundus. The retina is highly vascularized, meaning it contains a dense network of blood vessels, and it is part of the central nervous system. The retina is also largely transparent, allowing light to pass through and reach the photoreceptors. This transparency makes it possible to non-invasively capture detailed images that include the blood vessels and features of the central nervous system. These images can provide valuable information about the health of the vascular and nervous systems.

Multispectral and hyperspectral fundus imaging techniques have increasingly been used for diagnostic and other purposes. These techniques involve capturing images of the fundus and retina at different wavelengths of light, where the different wavelengths provide different spectral responses based on the features of the blood vessels and other structures in the fundus. These wavelength-specific images allow for more detailed analyses of the fundus/retina, including the detection and diagnosis of a wide range of ocular and systemic diseases, such as diabetes, cardiovascular diseases, neurological disorders like Alzheimer's disease (AD), organ-specific diseases, and the like.

Specially designed multispectral or hyperspectral cameras capture images of an object across a range of wavelengths by taking a series of images at different wavelengths (e.g., using bandpass filters or other techniques), and then combining the series of images into a single data “cube” that contains both spatial and spectral information in three dimensions (two spatial and one spectral). In fundus imaging applications, the captured reflectance spectrum is influenced by the molecular content (e.g., hemoglobin, melanin), cellular arrangement (e.g., capillaries, nerve fiber layer), and density/thickness (e.g., neurodegeneration) of the tissue at the various wavelengths of the spectrum.

### SUMMARY

Techniques described herein involve capturing and correcting multispectral or hyperspectral cubes containing retinal image data to increase the accuracy and effectiveness of diagnoses or other analyses based on the retinal images. The raw cubes produced by different cameras may have variations in pixel intensities due to differences in the spatial-spectral response of the camera, distortions caused by the eye, and other factors. In particular, the camera's light source, the transmission of the optical elements of the camera, the reflectance of the retina, and the spectral sensitivity of the camera sensor, among other factors may all impact the retinal image, making analysis more difficult. Correcting for these variations and distortions may help to ensure that the images of a given subject are easier to analyze as well as comparable across different cameras.

Accordingly, techniques described herein may include reflectance calibration and/or normalization of multispectral or hyperspectral image data by correcting the intensity values measured for each pixel and each spectral channel (or a subset thereof) so that they more accurately measure the true reflectance value of the tissue being imaged. These techniques may be performed using a calibrated reference standard as a reference point.

Previous work on normalization/calibration methods for fundus imaging may be insufficient to accurately correct retinal images, for example to identify subtle spatial-spectral features indicative of a disease. In particular, previous techniques may not account for several important factors that affect the accuracy of a captured cube of image data. Accordingly, techniques described herein include improved methods for multispectral or hyperspectral image data correction.

According to some embodiments of the present disclosure, a computer-implemented method, system configured to perform the method, and computer-readable medium including instructions for carrying out the method is disclosed. The method may include processing fundus imaging data to generate a calibrated eye measurement. The method includes receiving fundus imaging data comprising a plurality of images of a fundus of an eye, wherein the fundus imaging data is captured using a multispectral camera configured to capture images corresponding to different spectral bands. The method further includes receiving reference imaging data comprising a plurality of images of a reference model of an eye, wherein the reference model is a physical artificial eye, wherein the reference imaging data is captured using the multispectral camera. The method further includes adjusting the fundus imaging data to match the reference imaging data or the reference imaging data to match the fundus imaging data to yield post-adjusting fundus imaging data and post-adjusting reference imaging data by performing one or more of: compensating for a manufacturing imperfection in the reference model; compensating for a field of view difference between the fundus imaging data and the reference imaging data; compensating for a diffusivity difference between the human eye and the reference model; or compensating for a spectral difference in the media between the reference model and the human eye. The method further includes generating a calibrated eye measurement based on processing the post-adjusting fundus imaging data and the post-adjusting reference imaging data.

In some embodiments, the adjusting comprises compensating for the field of view difference between the fundus imaging data and the reference imaging data by: detecting a size difference and a position difference between the fundus imaging data and the reference imaging data; and transforming one of the fundus imaging data or the reference imaging data to remove the size difference and the position difference. In some of these embodiments, the method further includes detecting a first field of view of the fundus imaging data and a second field of view of the reference imaging data by detecting which pixels of the corresponding imaging data are associated with an intensity value that is greater than a threshold level.

In some embodiments, the adjusting comprises compensating for the diffusivity difference between the human eye and the reference model by: quantifying the diffusivity difference between the human eye and the reference model on a wavelength-by-wavelength basis; and for each wavelength of a set of one or more wavelengths, correcting the diffusivity difference between a fundus image associated with the wavelength and a reference image associated with the same wavelength by blurring whichever image is associated with a lesser diffusivity to match the corresponding image. In some of these embodiments, quantifying the diffusivity difference comprises using predetermined wavelength-specific diffusivity factors. Additionally or alternatively, the blurring comprises using a gaussian filter with a window size that is dependent on the corresponding wavelength.

In some embodiments, the adjusting comprises compensating for the manufacturing imperfection in the reference model by: for each wavelength of a set of one or more wavelengths captured by the multispectral camera, combining a plurality of images of the reference model taken from different orientations to generate a combined image for the corresponding wavelength. In some of these embodiments, the adjusting further comprises, for each wavelength of the set of one or more wavelengths, applying at least one edge-preserving filter to the corresponding averaged image, wherein the at least one edge-preserving filter comprises a median filter and a non-local means denoising algorithm.

In some embodiments, the adjusting comprises compensating for the spectral difference in the media between the reference model and the human eye by: for each wavelength of a set of one or more wavelengths captured by the multispectral camera, applying a corresponding correction coefficient for the human eye.

In some embodiments, the fundus imaging data is captured by the multispectral camera using a rolling shutter acquisition, wherein the normalizing further comprises: determining, for one or more horizontal rows of the fundus imaging data, a central wavelength; and removing a vertical spectral gradient in the fundus imaging data by interpolating between different images of the fundus imaging data for each of the one or more horizontal rows based on the corresponding central wavelength.

In some embodiments, adjusting the fundus imaging data to match the reference imaging data or the reference imaging data to match the fundus imaging data further comprises compensating for temporal light fluctuations in the fundus imaging data and the reference imaging data by adjusting based on an illumination power measurement to perform a power correction. In some of these embodiments, compensating for temporal light fluctuations in the fundus imaging data and the reference imaging data further comprises using dark image subtraction, wherein the dark image subtraction comprises subtracting at least one dark image captured using the multispectral camera.

In some embodiments, adjusting the fundus imaging data to match the reference imaging data or the reference imaging data to match the fundus imaging data further comprises compensating for parasitic reflections of optics of the multispectral camera in the fundus imaging data and the reference imaging data using baseline imaging data, wherein the baseline imaging data is captured using the multispectral camera, wherein the baseline imaging data comprises a plurality of images of a light trap.

In some embodiments, the method further comprises receiving a selection of a subset of the fundus imaging data, wherein the subset specifies one or more of a subset of wavelengths or a subset of pixels, wherein the adjusting and the generating are limited to the subset of the fundus imaging data.

These features, along with many others, are discussed in greater detail below.

### BRIEF DESCRIPTION OF THE DRAWINGS

The present disclosure is described by way of example and not limited in the accompanying figures in which like reference numerals indicate similar elements and in which:

FIG. 1 illustrates an example environment including an image correction system for carrying out the techniques described herein.

FIG. 2 illustrates an example cube of image data comprising images according to the techniques described herein.

FIG. 3 illustrates an example method for correcting eye measurements according to the techniques described herein.

FIG. 4 illustrates an example method for generating baseline imaging data according to the techniques described herein.

FIGS. 5A-B illustrate example data generated by adjusting reference image data to correct for structural imperfections of a reference eye model according to techniques described herein.

FIG. 6 illustrates example data generated by adjusting eye measurement and reference data to have matching fields of view according to techniques described herein.

FIG. 7 illustrates example data generated by a process for correcting diffusivity in imaging data according to techniques described herein.

FIG. 8 illustrates an example method for correcting eye measurements according to the techniques described herein.

FIG. 9 illustrates an example image correction system for carrying out the techniques described herein.

### DETAILED DESCRIPTION

Techniques described herein improve existing reflectance calibration methods for retinal cameras to enable more accurate analysis of retinal image data. Several improved techniques are described herein. In some embodiments, multispectral or hyperspectral retinal cubes may be corrected based on differences in light scattering properties (e.g., diffusivity) between the eye and a reference material. Additionally or alternatively, multispectral or hyperspectral retinal cubes may be corrected based on discrepancies in size and position of a reference measurement with respect to an eye measurement. Additionally or alternatively, multispectral or hyperspectral retinal cubes may be corrected based on the presence of an intraocular lens or some other condition that causes a spectral difference within a subject's eye as compared to a typical human eye. Additionally or alternatively, multispectral or hyperspectral retinal cubes may be corrected to remove a gradient created by a rolling shutter sensor acquisition method. These improvements and the other features described herein, whether used alone or in any combination, allow for more reliable and accurate analysis of retinal image data captured with multispectral or hyperspectral cameras.

The techniques described herein improve the state of the art in fundus imaging and analysis by providing novel image processing methods that calibrate or normalize the reflectance of the color channels of fundus images captured with multispectral or hyperspectral fundus cameras. In particular, the techniques described herein may correct the intensity of each pixel and each spectral band (or a subset thereof) of a multispectral cube to better correspond to the true reflectance of the retina and to permit more accurate quantitative analysis of the images, thereby allowing the extraction of spatial-spectral features that may be useful to detect signs of a disease having a manifestation in the fundus.

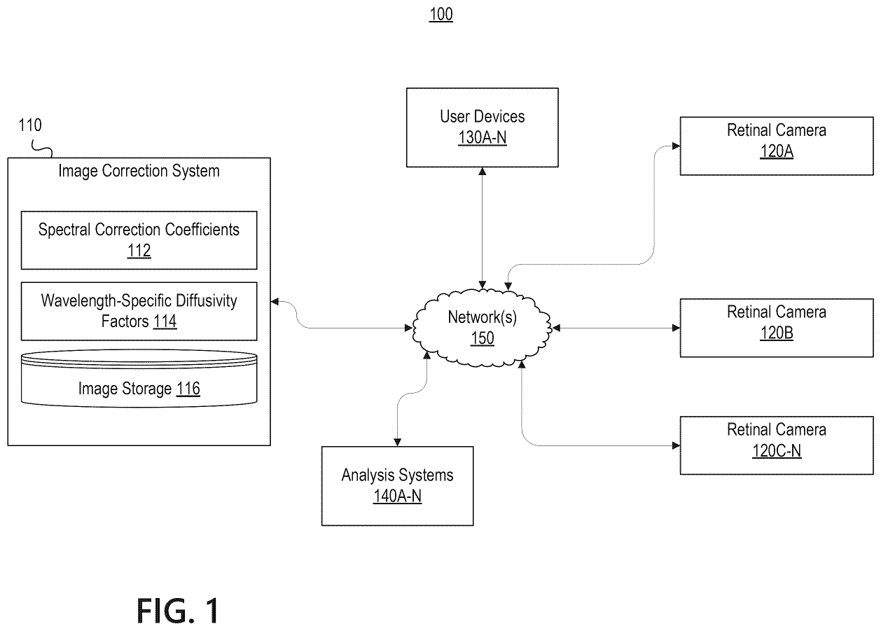

FIG. 1 illustrates an example environment 100 including a plurality of devices that may be used for carrying out the techniques described herein. The environment 100 may include an image correction system 110, a plurality of (hyper-or multi-spectral) retinal cameras 120A-N, one or more user devices 130A-N, and one or more analysis systems 140A-N in communication via one or more networks 150. It will be appreciated that the network connections shown are illustrative and any means of establishing communications links between the various devices and systems may be used, including direct cabling or other peer-to-peer communications instead of or in addition to the one or more networks 150. The existence of any of various network protocols such as TCP/IP, Ethernet, FTP, HTTP and the like, and of various wired or wireless communication technologies such as USB, GSM, CDMA, Wi-Fi, and LTE, is presumed, and the various computing devices described herein may be configured to communicate using any of these communication protocols or technologies.

The image correction system 110 may store various data for performing the various functions described herein. In general, the image correction system 110 may provide functionality for processing image data received from the various retinal cameras 120A-N as described herein. For example, the image correction system may be configured to receive multispectral or hyperspectral cubes comprising retinal scans, reference image data (e.g., white images, black images, images of a reference eye, etc.), and the like from a first retinal camera 120A, and to store such data in association with an identifier of the first retinal camera 120A, patient data and/or metadata for the patient associated with the retinal cube, and the like. The image correction system may store various data for correcting the multispectral or hyperspectral cubes, including spectral correction coefficients 112, wavelength-specific diffusivity factors, and the like, which are described in more detail below. The image correction system 110 may also store the data cubes, including metadata and any associated identifiers, in the image storage 116. Similarly, the image correction system may be configured to receive and store similar or different data cubes from each of the remaining retinal cameras 120B-N along with an identifier for the corresponding retinal camera that captured the data, corresponding patient data and/or metadata, etc. In embodiments, different retinal cameras 120 may be different types of camera (e.g., different manufacturer/make/model/etc.) and/or may capture different types of data (e.g., multispectral cubes vs. hyperspectral cubes).

In embodiments, although the image storage 116 is illustrated as being a component of the image correction system 110, in other embodiments the image storage 116 may be a part of other systems. For example, any data described herein may be stored in cloud storage, stored at a separate server device connected to the image correction system 110, and/or the like.

The image correction system 110 may perform various image correction methods on the cubes stored in image storage 116. For example, the image correction system 110 may correct retinal images data of a first cube received from a first retinal camera 120A using various other image data captured by the same retinal camera 120A using the techniques described in more detail below. The image correction system 110 may then store the corrected retinal image data in image storage 116 along with an identifier of the first retinal camera, patient data and/or metadata for a corresponding patient, etc. Similarly, the image correction system 110 may correct retinal image data received from any of the other retinal cameras 120B-N using reference image data that was captured by the same retinal camera 120B-N, and may store the corrected retinal cube in storage 116 along with an identifier of the corresponding retinal camera, corresponding patient data and/or metadata, etc.

The user devices 130A-N may be used by various users to interact with the image correction system 110, view uncorrected retinal image data, view corrected retinal image data, edit various data, generate analyses, and/or the like. For example, one user may use a first user device 130A to view a first corrected multispectral cube associated with a first patient, another user may use a second user device to view a second corrected multispectral cube associated with a second patient, and the like. In embodiments, the image correction system 110 may restrict access to the retinal cube data (e.g., in accordance with medical data privacy laws or other rules that may vary by jurisdiction) to certain user devices based on authorization credentials provided by a user using the user device 130. The user devices 130A-N may include mobile devices (e.g., smartphones, laptops, tablets), wearable devices (e.g., smartwatches), other computing devices (e.g., desktop computers, servers), and/or any other devices that may access and interact with the image correction system 110.

The analysis systems 140A-N may include automated systems that may use the uncorrected image data, corrected image data, and/or other data described herein to perform automated analyses. For example, the analysis systems may use machine learning, machine vision, or other automated approaches to perform automated analysis, generate diagnostic information, and/or the like. In embodiments, the automated analyses and/or diagnostics generated by the analysis systems may be provided by the analysis systems 140A-N to the image correction system 110 and/or to the user devices 130A-N. In embodiments, the image correction system 110 and/or the analysis devices 140A-N may require authorization from a particular user device 130 to make the analyses and/or diagnostics available to the user device 130.

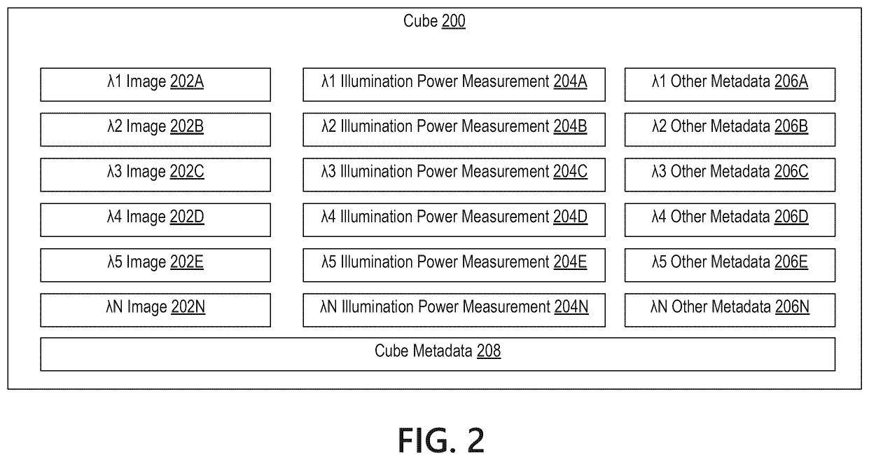

FIG. 2 illustrates an example multispectral or hyperspectral cube 200 comprising a series of images 202 (e.g., retinal images), a plurality of illumination power measurements 204 for each image, and a plurality of other metadata 206 for each image. The cube may also comprise cube metadata 208. For convenience, the cube 200 will be referred to in some embodiments only as a multispectral cube, but it should be understood that a hyperspectral cube may also be used instead of a multispectral cube for any embodiments described herein. As shown in FIG. 2, the cube 200 is a data structure that comprises a series of images captured at different wavelengths. Although the illustrated cube 200 shows only a few images for simplicity, in embodiments, a hyperspectral cube may include a larger number of images (e.g., tens, hundreds, or thousands) that may be captured across a range of wavelengths, which may be overlapping. By comparison, a multispectral cube may include a smaller number of narrowband images (e.g., a few dozen or fewer) that may be captured across a different range of wavelengths, which may often include spectral gaps such that the cube includes spaced spectral bands. Accordingly, a hyperspectral cube may have a higher spectral resolution and a continuous spectral band, whereas a multispectral cube may be faster and easier to acquire, process, and analyze. Due to these differences, hyperspectral and multispectral cubes may each be preferable in different circumstances.

It should be noted that the cube 200 is only an example of a data cube, but other data cubes may have different data structures. For example, instead of storing separate images 202A-N, the data cube may store a single three-dimensional data structure where two dimensions are spatial dimensions and a third dimension is a spectral dimension. Additionally or alternatively, the cube 200 may separately store other “slices” of the data cube, such as a series of images where each image has a first spatial dimension and a second spectral dimension, and each image corresponds to a different row or column along a second spatial dimension.

As shown at FIG. 2, the example cube 200 comprises a series of images 202, where a first image 202A may correspond to a first wavelength λ1, a second image 202B may correspond to a second wavelength λ2, and so on. The cube 200 may be generated by a multispectral retinal camera 120 that captures the images and combines the images into a single data cube 200 that includes both spatial and spectral information. In other words, the reflectance spectrum of each pixel of each image may indicate the molecular makeup, cellular arrangement, and/or density of the retinal tissue at the corresponding wavelength, as well as other influences that may be removed by the correction process as described in more detail below. Although an image may be referred to as corresponding to a particular wavelength, in practice each image in the cube may include information captured within a specific band of wavelengths that includes the particular wavelength (e.g., the term “wavelength” may refer to a representative wavelength within a band). The bands may be relatively broad and/or non-overlapping (e.g., for multispectral cubes) or relatively narrow and/or contiguous or overlapping (e.g., for hyperspectral cubes).

In embodiments, the cube 200 may further comprise an illumination power measurement 204 for each wavelength, which may be a measurement of the illumination power that was generated by the illumination light of the retinal camera in that wavelength (or band of wavelengths). The power measurements may be different due to temporal light fluctuations during the capture as well as the illumination light's output spectrum. Each image of a cube may also include various other metadata 206. The metadata may include data indicating the type of image, the wavelength or wavelength band for the image, retinal camera settings used to capture the image (e.g., focus amount), and the like. In embodiments, the cube 200 may further include cube metadata 208 that may include data about the type of cube (e.g., an eye measurement cube, a reference cube, a baseline cube, etc.), an identifier of a camera that captured the cube, patient information associated with the cube, and the like.

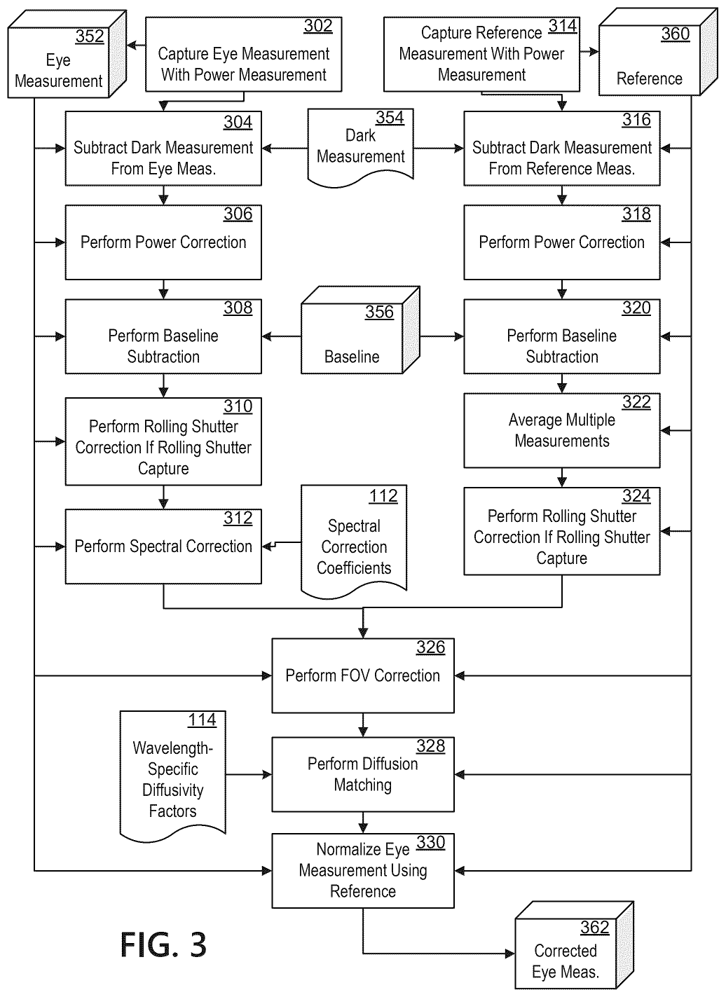

FIG. 3 illustrates an example method of capturing and correcting a multispectral cube 352 comprising retinal image data that provides an eye measurement in order to generate a cube 362 comprising a corrected eye measurement. The corrected eye measurement cube 362 may include image data with various influences removed, such as light intensity fluctuations, parasitic reflections, reflectance properties of the eye (e.g., diffusivity correction), spectral effects of an intraocular lens or other condition, etc.

At step 302, a retinal camera 120 (e.g., a first retinal camera 120A) may capture a first multispectral cube 352 that provides a retinal eye measurement (e.g., the captured image data is of a human patient's retina). In some example embodiments, the multispectral cube includes a power measurement for each wavelength or band of wavelengths captured and stored as part of the multispectral cube. The power measurements may vary spectrally. The retinal camera 120 may transmit the captured cube to the image correction system 110 (e.g., across a network 150 or other link, such as a cable), which may receive the cube for further processing.

In some cases, the image correction system 110 may correct the entire cube 352 using the method of FIG. 3. However, in other cases, the image correction system 110 may select a subset of the cube 352 for correction. For example, certain wavelengths may be of interest for a particular analysis or use case. Thus, the subset may include one or more of the images 202 of a particular cube 200 (e.g., if wavelength λ3 is of interest, the subset may include only image 202C; if wavelengths λ2-λ4 are of interest; the subset may include only images 202B-D) The subset may include a single image (e.g., corresponding to a particular wavelength) or a plurality of images (e.g., corresponding to one or more particular ranges of wavelengths, which may be continuous or discontinuous). Additionally or alternatively, certain regions of an image (e.g., subsets of the pixels of each image of the cube) may be of interest for a particular analysis or use case. Accordingly, the subset may include only certain region(s) of each image (or a portion of the images if only certain wavelengths are of interest). Thus, as used herein, a subset of a cube 352 may refer to some portion of the cube 352, whether the portion omits some or all of the wavelength-specific images 202 and/or whether the portion omits some or all of the pixels of each image 202. In these cases, the image correction system 110 may correct only the subset of the cube 352 to improve efficiency by reducing the amount of data that must be processed.

At step 304, the image correction system 110 may begin correcting the eye measurement cube 352 by subtracting a dark measurement 354 from the eye measurement cube 352. Subtracting the dark measurement from the eye measurement may help to remove noise or background signal that may be present in the image data, allowing for more accurate analysis of the eye tissue. The dark measurement 354 is a reference measurement that may be taken by the same retinal camera that captured the eye measurement 352 (e.g., retinal camera 120A). The dark measurement may be taken with the light source of the retinal camera turned off. The dark measurement may be captured using the same exposure time as the eye measurement. In embodiments, the dark measurement may be generated by capturing several dark images (e.g., images of the dark calibration target with the illumination source off) using the given exposure time and then averaging (e.g., using a mean, median, or mode average) or otherwise combining them to create a dark measurement. Additionally or alternatively, the dark measurement may be a cube with the same or similar data structure as the eye measurement cube 352.

To subtract the dark measurement from the eye measurement, the image correction system 110 may subtract each pixel value of the dark measurement 354 from the corresponding pixel value of the multispectral cube 352. In cases in which only a subset of the cube 352 is being normalized, only the pixel values corresponding to the subset may be corrected by subtracting the corresponding dark pixel value. For example, the image correction system may take a first retinal image 202A of a multispectral cube (or some other 2-d slice of the three-dimensional cube, or a selected pixel of the multispectral cube, depending on the data structure of the cube), and proceed to reduce the value of a selected first pixel by the value of the corresponding pixel of the dark measurement, reduce the value of a second selected pixel by the value of its corresponding dark pixel, and so on. The image correction system may then proceed in a similar way to correct all (or a subset of) of the retinal image data (e.g., each of the images 202B-N). The image correction system may store the dark-corrected eye measurement cube 352 in image storage 116 and proceed to the next step.

The image correction system 110 may proceed with the method at step 306 by performing a power correction on the dark-corrected eye measurement using the power measurements 204 that were stored as metadata of the eye measurement cube 352. The power measurement correction may correct for variations in the intensity of the light source used by the retinal camera, including variations across the different wavelengths of the spectral dimension of the image data. The image correction system 110 may take the output of step 304 (the dark-corrected eye measurement cube) and divide the pixel values of each image (or a subset thereof, if only a subset of the pixel values and/or images are being corrected) by the power measurement factor stored as metadata for the corresponding image, thus removing the influence of the power variations from the resulting image data. For example, the image correction system 110 may divide each pixel value (or a subset thereof) by the power measurement corresponding to that pixel (e.g., using a first power correction measurement 204A to correct each pixel of the first image 202A, using a second power correction measurement 204B to correct each pixel of the second image 202B, and so on), to generate a dark-corrected and power-corrected eye measurement.

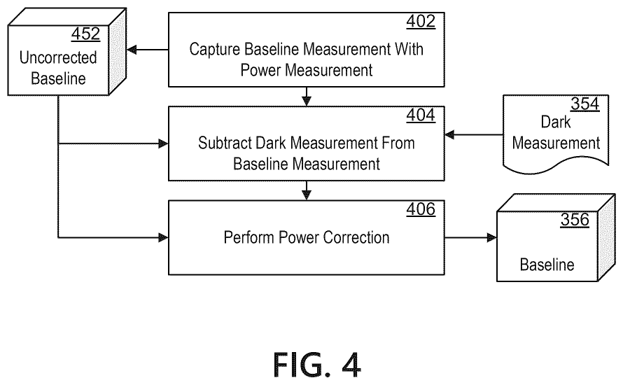

At step 308, the image correction system 110 may perform a baseline cube subtraction to further correct the eye measurement for internal parasitic reflections of the retinal camera. The baseline cube 356 is a multispectral reference cube that may be obtained using the same settings that were used to capture the eye measurement cube 352 and may be corrected in the same way. The retinal camera may have previously captured the baseline cube 356 (e.g., at a time prior to the method of FIG. 3) with the light source of the retinal camera activated and the retinal camera focused on a light trap (e.g., a beam dump). The baseline cube 356 can therefore capture the effect of any parasitic reflections generated by the interaction of the light source with the internal optics of the retinal camera. The baseline cube may have been captured using the process shown at FIG. 4, described immediately below.

Turning to FIG. 4 to explain the process of generating the baseline cube 356, at step 402 the same retinal camera 120 used to capture the eye measurement at step 302 (e.g., a first retinal camera 120A) may capture a multispectral cube 452 that provides an uncorrected baseline measurement (e.g., the captured image data is of a beam dump/light trap) for that camera. In embodiments, the uncorrected baseline cube 452 includes a power measurement for each of a series of image captured and stored as part of the cube, as shown at FIG. 2. In general, the cube 452 may be captured using the same camera and settings as the first multispectral cube 352 that provides the eye measurement, and may store the image data in the same format. After capturing the cube 452, the retinal camera 120 may transmit the cube 452 to the image correction system 110, which in turn receives the cube for correction.

At step 404, the image correction system 110 may subtract the dark measurement 354 from the baseline measurement provided in the cube 452. The dark measurement may be the same dark measurement used for step 304 as described above, and the subtraction may proceed in the same way as described above for step 304. Similarly, at step 406, the image correction system 110 may perform a power correction using the power measurements that were stored as metadata of the cube 452. Again, the power correction may proceed as described above for step 306, with the image correction system 110 using the baseline's power measurements to correct each corresponding pixel of the cube. The result may be a baseline cube 356 that is dark-corrected and power-corrected.

Returning to step 308 of FIG. 3, the image correction system 110 may subtract the baseline cube 356 from the corrected eye measurement cube as output by step 306 to obtain an eye measurement that is baseline-corrected, dark-corrected, and power-corrected. As above, the image correction system 110 may correct the pixel values of the eye measurement cube by subtracting each pixel value of the baseline cube (or a subset thereof, if only a subset of the pixel values are being corrected) from the corresponding pixel value of the eye measurement cube. The image correction system 110 may then store the baseline-corrected, dark-corrected, and power-corrected eye measurement cube 352 for further processing. It should be noted, however, that in some cases one or more of the baseline correction, dark correction, and power correction may be omitted because one or more of these techniques may not be necessary for all applications and/or for all cameras. For example, the baseline correction may be omitted without significantly affecting accuracy if parasitic reflections from the optical elements of the retinal camera will fall onto known pixels that are purposedly excluded from the calibration. Similarly, the dark correction may be omitted without significantly affecting accuracy if the intensities of the eye measurement and the reference cube (described in more detail below) across the whole camera sensor and for all wavelengths are a few orders of magnitude higher than the dark intensities, such that the measurement uncertainty is higher than the dark values.

After one or more of the above-described corrections is applied, one or more further corrections may be applied, including a rolling shutter correction (e.g., if a rolling shutter capture technique was used), an intra-ocular lens correction (if a patient has an intra-ocular lens implanted), a field of view (FOV) correction, and diffusion matching. Some of these additional techniques may not be used in all circumstances and for all applications, so the method of FIG. 3 should be understood as describing one example embodiment of a correction process.

At step 310, the image correction system 110 may perform a rolling shutter correction if a rolling shutter capture was used to obtain the multispectral cube including the eye measurement. In a rolling shutter capture mode, the retinal camera captures a horizontal row of pixels for an image at the same time, then moves onto the next horizontal row. Thus, different rows of the image data are captured at different times. This may result in a vertical gradient appearing in the image data due to temporal changes in the illumination light wavelength as the image is progressively captured. In other words, the first row may be captured at a wavelength that is a first delta amount different from a reference wavelength for a particular image, the second row may be captured at a different wavelength that is a second delta amount different from a reference wavelength for that same image, and so on.

The image correction system 110 may perform the rolling shutter correction by interpolating across subsequent images of the multispectral cube in order to reconstruct monochromatic images (e.g., an image where each pixel corresponds to the same wavelength). For example, the image correction system 110 may determine a central wavelength for each horizontal row of the fundus imaging data (where the central wavelength may vary by some difference from the wavelength corresponding to the image). The image correction system 110 may then remove the vertical spectral gradient in the fundus imaging data by interpolating between different images of the fundus imaging data for each horizontal row based on the determined central wavelength.

In embodiments in which only a subset of the wavelength-based images of the cube 352 are being corrected, the image correction system 110 may perform the rolling shutter correction on only the subset of the images, which may require the use of an additional image outside the subset of the images. For example, if images 202B-D (corresponding to λ2-4) are being corrected, then the image correction system 110 may use images 202A-D to perform the rolling shutter correction of images 202B-D (e.g., because the rolling shutter correction for image 202B may interpolate based on images 202A-B, the rolling shutter correction for image 202D may interpolate based on images 202C-D, etc.). Accordingly, the image correction system 110 may use images outside a selected subset for rolling shutter correction of the subset.

At step 312, the image correction system 110 may perform a spectral correction if necessary or desirable. A spectral correction may be necessary or desirable if the patient has an intra-ocular lens (IOL) implant, cataracts, or some other condition in the ocular medium that may cause a spectral difference in the patient's eye versus a typical human eye. For example, an IOL (a synthetic lens that may be used to treat various conditions) or some other condition may cause the eye to reflect light spectrally differently from a natural lens, which may make comparison of retinal images and diagnoses or other analyses of retinal images more difficult and less accurate. The image correction system may apply spectral correction coefficients 112 (e.g., IOL correction coefficients) to adjust each of the various images of the eye measurement cube (or a subset of the images, if only a subset are being corrected) by a different amount. For example, a first spectral correction coefficient 112 may be used to adjust each of the pixel values (or a subset thereof) of the first image 202A of the multispectral cube (e.g., by multiplying the pixel value by the correction coefficient), a second spectral correction coefficient 112 may be used to adjust each of the pixel values (or a subset thereof) of the second image 202B of the multispectral cube, and so on.

The image correction system 110 may contain different sets of spectral correction coefficients 112, and may select and use one of the different sets to correct a particular eye measurement based on various factors. For example, if the patient has an IOL, the image correction system 110 may select a set of spectral correction coefficients provided by the IOL manufacturer that have been tuned to a specific IOL implant provided by that manufacturer. However, if the manufacturer does not provide a set of correction coefficients for a specific IOL, or if the specific IOL is not known, or if some other condition such as cataracts are present, a generalized set of correction coefficients may be selected and used based on various factors such as a color of the IOL (e.g., blue blocking filter, UV blocking filter, etc.) or other known properties of the IOL or other condition, which may be observed by a clinician or camera operator (or determined from patient data) and input to the image correction system 110 (e.g., at the time of image capture or otherwise) or a retinal camera 120. The properties may be stored (e.g., by the retinal camera 120 or image correction system 110) as metadata of the cube 352 or otherwise stored in association with the cube 352. Additionally or alternatively, a particular spectral correction coefficient 112 for each image 202 may be selected based on the wavelength associated with a corresponding image 202 (or band of wavelengths, bandwidth, etc.).

After performing the rolling shutter correction and/or spectral correction, the eye measurement cube 352 may be ready for FOV correction at step 326 and/or diffusion matching at 328. However, as a pre-condition, the image correction system 110 may correct a reference cube 360 using some or all of the same techniques used to correct the eye measurement cube (e.g., using a process shown in steps 314-324 of FIG. 3). Steps 314-324 are described below, but may be performed before or after steps 302-312. In cases in which only a subset of the eye measurement cube 352 is being normalized, steps 314-324 may be used to correct a corresponding subset of the reference cube 360 prior to FOV correction at step 326. Alternatively, the entirety of the reference cube 360 may be corrected at steps 314-324 prior to FOV correction at step 326. The latter method may be more appropriate when a subset of the pixels of each image (e.g., only a portion of each image) is being corrected (e.g., because the pixels of the reference cube and the pixels of the measurement cube may not match up prior to FOV correction). The former method may be more appropriate in other cases to reduce unnecessary processing.

At step 314, the same retinal camera 120 used to capture the eye measurement cube 352 may be used to capture the reference cube 360. The retinal camera 120 may use the same settings (e.g., the same wavelengths, exposure times, etc.) to capture the reference cube 360 as the eye measurement cube 352. The reference cube 360 may be captured by focusing the retinal camera 120 on a physical eye model mimicking the light optical propagation in the anterior segment of the human eye, where the “fundus” of the physical eye model is composed of a reference material of known reflectivity (e.g., a white or gray material). The reference cube 360 may include a power measurement for each wavelength of the image data, as shown for FIG. 2. After capturing the cube 360, the retinal camera 120 may transmit the cube 360 to the image correction system 110, which in turn receives the cube 360.

As compared to the eye measurement cube 352, the reference cube 360 may comprise additional sets of images such that multiple images (e.g., 36 images) are captured for each wavelength. For example, a first set of images 202A may be captured for a first wavelength, a second set of images 202B may be captured for a second wavelength, and so on. Multiple images may be captured for each wavelength due to the use of an averaging process for the reference cube, as explained in more detail below for step 322. Each image of a set of images may be taken at a different angle/rotation/orientation, which allows the averaging process to remove structural defects, as described in more detail below.

At step 316, the image correction system 110 may subtract the dark measurement 354 from the reference measurement provided in the cube 360. The dark measurement may be the same dark measurement used for step 304 as described above, and the subtraction may proceed in a similar way as described above for step 304. Similarly, at step 318, the image correction system 110 may perform a power correction using the power measurements that were stored as metadata of the cube 360. Again, the power correction may proceed as described above for step 306, with the image correction system 110 using the reference cube's power measurements to correct each corresponding pixel of the series of images (or a subset thereof) within the cube 360. The result may be a reference cube 360 with a series of images for each wavelength (or a selected subset of wavelengths), where the series of images is dark-corrected and power-corrected. If the dark correction or power correction was omitted for the eye measurement cube 352 (e.g., if steps 304 and/or 306 were skipped), then in some cases the corresponding steps may also be skipped for the reference cube 360 so that the eye measurement cube 352 and the reference cube 360 are corrected in the same way. Additionally or alternatively, in some cases (e.g., depending on the retinal camera in question, the reference eye in question, the eye being measured, etc.), certain corrections may be applied only for the measurement cube and/or only for the reference cube.

At step 320, the image correction system 110 may perform a baseline subtraction by subtracting the baseline cube 356 from the dark-and power-corrected reference cube 360. The baseline cube 356 subtraction may operate in a similar way as explained above for step 308, with the same baseline cube 356 being used for both steps 308 and 320. If the image correction system 110 skipped the baseline subtraction for the eye measurement cube, the image correction system 110 may also skip the baseline subtraction for the reference cube 360 (e.g., if step 308 was skipped then step 320 may be skipped). The baseline cube 356 may, after step 320, comprise a series of images for each wavelength, where each of the images (or a subset thereof) is dark-corrected, power-corrected, and baseline-corrected.

At step 322, the image correction system 110 may generate an average of multiple reference images for each wavelength of the reference cube 360 (or a selected subset thereof). The image correction system 110 may use the averaging process to correct for structural defects of the eye model (e.g., spots, lines, etc. that show up in the retinal image due to structural features created by the manufacturing of the eye model). Because each image of the set of images for each wavelength of the reference cube may be taken at a different orientation (e.g., different angle and/or position with respect to the retinal camera), the image correction system 110 may average the different images of each set of images (e.g., so that each set of images results in a single averaged image, with the cube then containing a single averaged image per wavelength) to significantly reduce the effects of the structural features, which may show up as noise that affects various pixels of the averaged images. In embodiments, the image correction system 110 may then apply an edge-preserving filter, such as a median filter or a non-local-means denoising algorithm, to further reduce the noise corresponding to the structural defects from the averaged images.

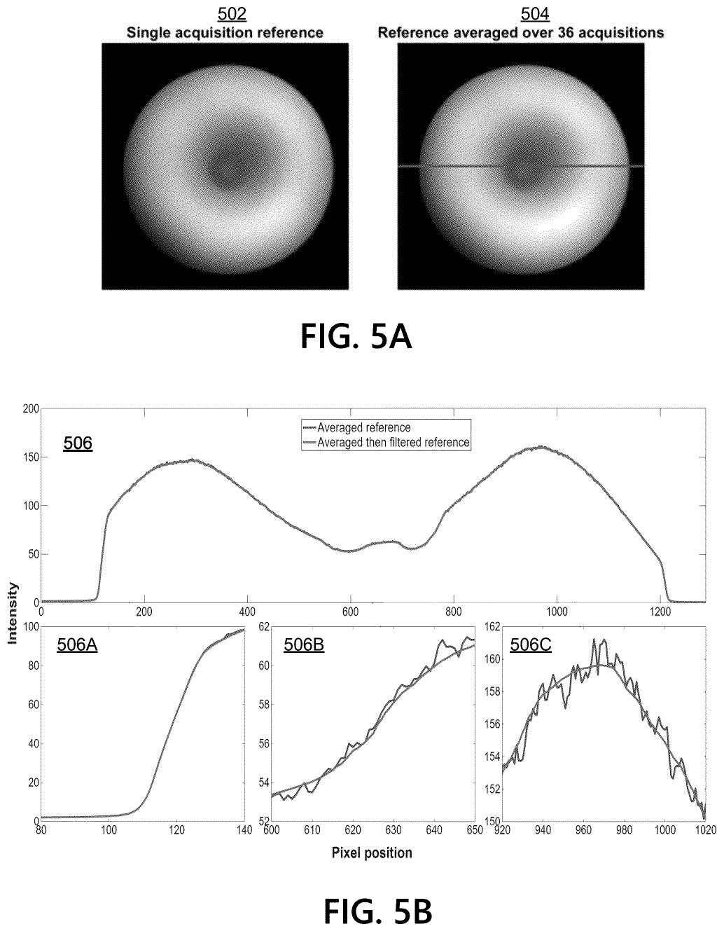

Example images and graphs illustrating the principles and effects of the averaging process are shown at FIG. 5. The first image 502 is an example of one of the set of reference images captured for a particular wavelength. As shown in FIG. 5, the example image contains various lines and other noise caused by the presence of structural defects in the eye model. The averaged image 504, by contrast, has a greatly reduced amount of this structural noise because it was generated by averaging 36 different images taken at various orientations.

The graph 506 shown at FIG. 5 shows the principles and effects of the filtering process that further removes noise from the averaged image. The graph 506 illustrates an example of the pixel intensity values for a single pixel row of an example averaged image and the corresponding pixel row of an example averaged then filtered image. The three sub-graphs 506A-C show zoomed in portions of the comparison graph in order to better reveal the differences between the two lines representing the first average image and the second averaged then filtered image. The noisier line corresponds to the averaged reference image and the smoother line corresponds to the averaged then filtered reference image, thus illustrating how the filtering step further smooths out the noise in the averaged image.

Returning to FIG. 3, at step 324, after performing the averaging process, the image correction system may take the resulting reference cube 360 and apply a rolling shutter correction if a rolling shutter capture method was used to capture the reference cube 360. The rolling shutter correction may occur in a similar way as described above for step 310, with the rolling shutter correction being applied to the corrected image data output by step 322.

At step 326, the image correction system 110 may use the eye measurement cube 352 and the reference cube 360 (with some or all of the above-described corrections applied, depending on whether a rolling shutter capture was used, whether an IOL is implanted, etc.) to perform a field of view correction. Because the eye model used for the reference may not perfectly match the eye used for the eye measurement (e.g., the eye model may be a different size from the eye and/or the eye model may be at a slightly different position or orientation with respect to the retinal camera as compared to the eye, requiring a different focus adjustment), the field of view with respect to the eye in the eye measurement cube 352 may be different than the field of view with respect to the eye model in the reference cube 360. This issue may be corrected either by adjusting the images within the eye measurement cube 352 so their FOV matches that of the images within the reference cube 360, or conversely by adjusting the images with the reference cube 360 so their FOV matches that of the images within the eye measurement cube 352. The adjustment may include a rescaling and/or a translation of one image onto the other (or some other type of image warping or adjustment) so that the FOVs match.

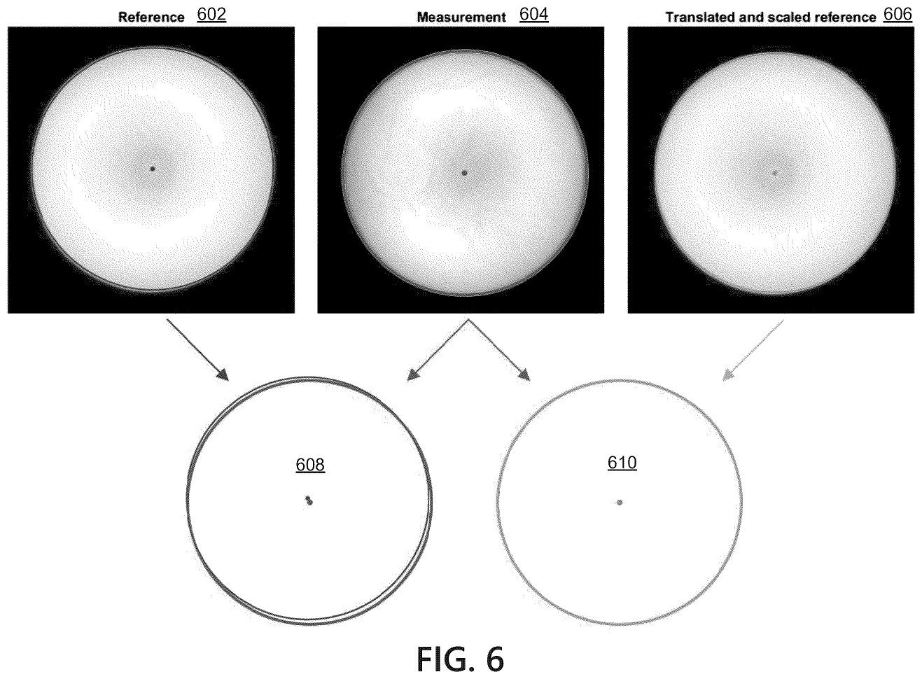

An example rescaling and translation as performed for two images by the image correction system 110 is shown at FIG. 6. In the example, a reference image 602 is translated and scaled to match the FOV of the eye measurement image 604, which yields a translated and scaled reference image 606. A first comparison 608 shows two different circles with two different center points to represent the two FOVs of the reference image 602 and the eye measurement image 604, which visibly differ as shown by the comparison. The second comparison 610 shows that the FOV of the measurement image 604 closely matches the FOV of the translated and scaled reference image 606, such that only a single circle is visible (illustrating the matching FOVs).

The image correction system 110 may translate and scale the reference images (or conversely the eye measurement images) on an image-by-image basis so that each image of the two cubes matches. For example, the image correction system 110 may translate and/or scale a first reference image corresponding to a first wavelength so that it matches a first eye measurement image corresponding to the first wavelength, translate and/or scale a second reference image corresponding to a second wavelength so that it matches a second eye measurement image corresponding to the second wavelength, and so on (e.g., assuming the images were captured sequentially). If the image data cube was captured in some other way (e.g., if spatial-spectral slices were captured sequentially), then the corresponding spatial-spectral slices of the two cubes may be compared and matched to account for any spectral variations in the FOV across the slices. If the FOVs of the images do not change spectrally, then the image correction system 110 may apply the same FOV correction (e.g., translating and/or scaling) across the entire cube.

Next, at step 328 the image correction system 110 may perform diffusion matching to correct for diffusivity differences between the eye model captured in the reference cube 360 and the human eye captured in the eye measurement cube 352. The diffusivity differences may be wavelength dependent. These diffusivity differences may manifest as different blurriness levels in one cube versus another. To perform the diffusion matching, the image correction system 110 may compare an eye measurement image to a reference image (e.g., where the images correspond to the same wavelength), and then may blur whichever image is less blurry to match the other image. When only a subset of the images and/or pixels are being corrected, the image correction system may compare then blur the corresponding subsets of each cube. Additionally or alternatively, the image correction system 110 may use stored wavelength-specific diffusivity factors 114 to blur the images (e.g., the reference images) or subsets thereof, as described in more detail below. The image correction system 110 may use a Gaussian filter or some other filter to increase a blur amount.

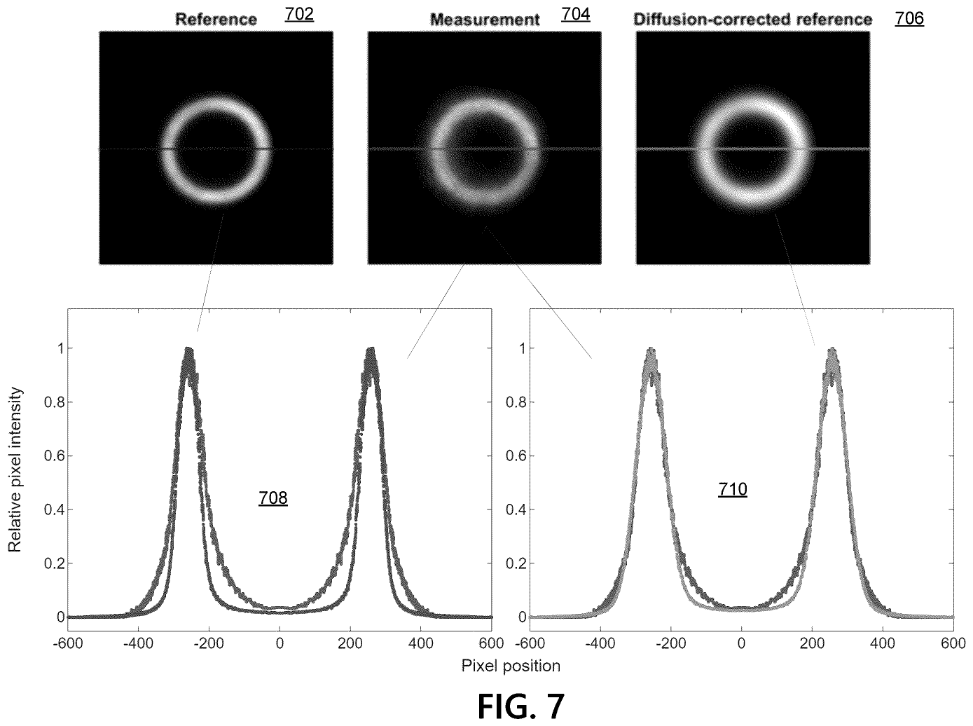

FIG. 7 illustrates an example of applying blur to correct diffusion difference between two images taken using a light ring. In this example, the light ring may be used to test the blur correction because it more easily shows diffusion differences between the reference image and the measurement image. In the example, the measurement image 704 is blurrier than the reference image 702, so the reference image may be diffusion corrected by blurring it, thus yielding a diffusion-corrected reference image 706 that more closely matches the diffusion of the measurement image 704. A comparison intensity graph 708 for a selected horizontal row of the reference image and the measurement image shows how the diffusivity difference manifests in differently shaped intensity curves. However, the post-correction comparison graph 710 shows how, after applying a Gaussian blur, the intensity curve of the measurement image and the diffusion-corrected reference image are much more similar.

In some embodiments, as shown at FIG. 3, the image correction system 110 may apply predetermined wavelength-specific diffusivity factors 114 to each image wavelength, where each wavelength-specific diffusivity factor 114 is determined prior to step 328 using a light ring comparison as shown at FIG. 7. For example, using light ring testing, the image correction system 110 may determine that a first amount of blur should be applied to the reference image for a first wavelength and may store a corresponding wavelength-specific diffusivity factor 114 for the first wavelength. Then, the image correction system 110 may determine that a second amount of blur should be applied to the reference image for a second wavelength and may store second amount of blur as a wavelength-specific diffusivity factor 114 for the second wavelength, and so on. At step 328, the image correction system 110 may use the wavelength-specific diffusivity factors 114 to correct the images of the reference cube 360 (e.g., by multiplying each pixel value by the corresponding wavelength-specific diffusivity factor), or a subset of the images. The use of the wavelength-specific diffusivity factors 114 may allow the image correction system 110 to correct for diffusivity without the need to compare blurriness between the actual measurement cube 352 and the reference cube 360, which may be difficult due to the presence of retinal tissue in the measurement image as well as other image differences.

At step 330, after performing some or all of the above-described corrections on the eye measurement cube 352 and/or the reference cube 360, the image correction system may use the reference cube 360 to normalize the eye measurement cube 352 to produce the corrected eye measurement cube 362. For example, the image correction system 110 may divide the pixel intensity values of the first image of the eye measurement cube 352 (as corrected using the above-described steps) by the corresponding pixel intensity values of the corresponding first image of the reference cube 360 (as corrected using the above-described steps) to generate the first image of the corrected eye measurement cube 362, then repeat for the second image, third image, and so on. In embodiments in which only a subset of the cube is corrected, the image correction system 110 may divide the pixel values of only the subset of the measurement cube by the corresponding pixel values of the reference cube. Because the pixel division operation removes matching structures, the image correction system 110 thereby removes the spatial-spectral response (e.g., illumination profile, spectral illumination intensity, spectral transmission of the optical elements, spectral response of the imaging sensor, etc.) of the imaging system from the eye measurement. From the above-described steps, the corrected eye measurement cube 362 therefore may have been corrected for temporal incident light fluctuations (e.g., using the dark and/or power corrections), internal parasitic reflections (e.g., using the baseline subtraction), a gradient caused by a rolling shutter capture, the presence of an inter-ocular lens, and the influence of the eye (after correcting the eye model reference to remove structural defects, match the FOV of the reference to the eye measurement, and diffusion match the reference to the eye measurement). Accordingly, with some or all of the above steps applied, various actors (e.g., automated systems and/or human doctors, analysts, etc.) may use the normalized eye measurement for more accurate diagnostics or comparisons. The image correction system 110 thereby enables improved clinical analyses and outcomes.

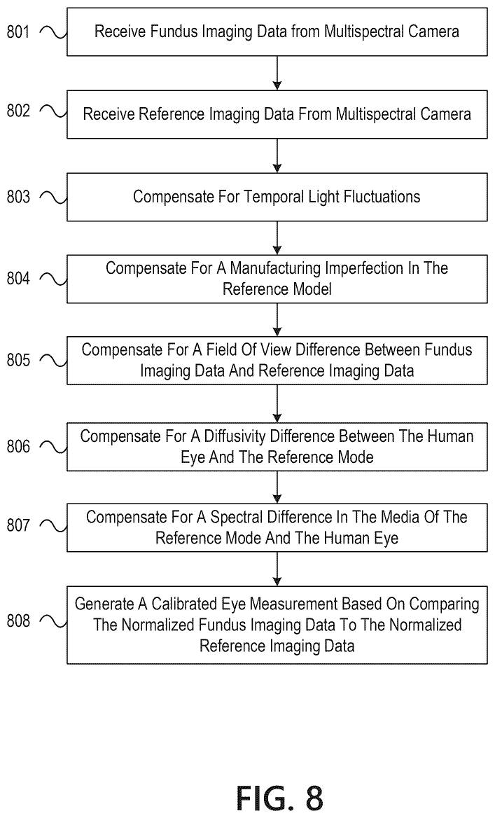

FIG. 8 illustrates an example method for processing fundus imaging data to generate a calibrated eye measurement according to one or more embodiments described herein. The example method of FIG. 8 may be performed by an image correction system. Although the method of FIG. 8 illustrates a particular example set of operations, it should be understood that in other examples, some or all of the operations may be omitted or performed in a different order than the one illustrated. At step 801, the image correction system may receive fundus imaging data comprising a plurality of images of a fundus of a human eye, wherein the fundus imaging data is captured using a multispectral camera configured to capture images corresponding to different spectral bands. At step 802, the image correction system may receive reference imaging data comprising a plurality of images of a reference model of an eye, wherein the reference model is a physical artificial eye, wherein the reference imaging data is captured using the multispectral camera.

The image correction system may then normalize the fundus imaging data and the reference imaging data. At step 803, the image correction system may compensate for temporal light fluctuations in the fundus imaging data and the reference imaging data using dark image subtraction, wherein the dark image subtraction comprises subtracting at least one dark image captured using the multispectral camera. The image correction system may then adjust the fundus imaging data to match the reference imaging data or the reference imaging data to match the fundus imaging data using one or more steps. At step 804, the image correction system may compensate for a manufacturing imperfection in the reference model. At step 805, the image correction system may compensate for a field of view difference between the fundus imaging data and the reference imaging data. At step 806, the image correction system may compensate for a diffusivity difference between the human eye and the reference model. At step 807, the image correction system may compensate for a spectral difference between a typical human eye and the measured human eye.

At step 808, the image correction system may then generate a calibrated eye measurement based on comparing the normalized fundus imaging data to the normalized reference imaging data.

The data transferred to and from various computing devices in the environment 100 may include secure and sensitive data, including personally identifiable information and patient data. Therefore, it may be desirable to protect transmissions of such data using secure network protocols and encryption, and/or to protect the integrity of the data when stored on the various computing devices. For example, a file-based integration scheme or a service-based integration scheme may be utilized for transmitting data between the various computing devices. Data may be transmitted using various network communication protocols. Secure data transmission protocols and/or encryption may be used in file transfers to protect the integrity of the data, for example, File Transfer Protocol (FTP), Secure File Transfer Protocol (SFTP), and/or Pretty Good Privacy (PGP) encryption. In many embodiments, one or more web services may be implemented within the various computing devices. Web services may be accessed by authorized external devices and users to support input, extraction, and manipulation of data between the various computing devices in the environment 100. Web services built to support a personalized display system may be cross-domain and/or cross-platform and may be built for enterprise use. Data may be transmitted using the Secure Sockets Layer (SSL) or Transport Layer Security (TLS) protocol to provide secure connections between the computing devices. Web services may be implemented using the WS-Security standard, providing for secure SOAP messages using XML encryption. Specialized hardware may be used to provide secure web services. For example, secure network appliances may include built-in features such as hardware-accelerated SSL and HTTPS, WS-Security, and/or firewalls. Such specialized hardware may be installed and configured in the environment 100 in front of one or more computing devices such that any external devices may communicate directly with the specialized hardware.

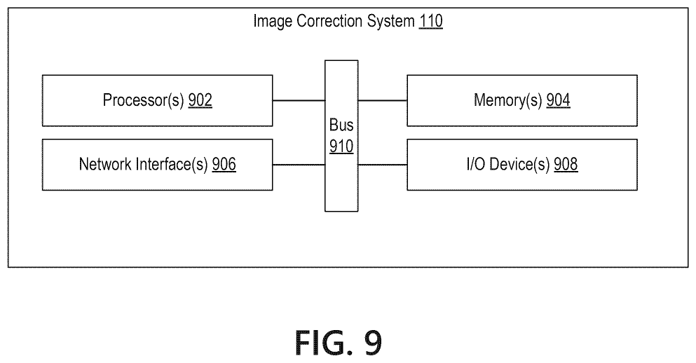

FIG. 9 illustrates an example image correction system 110 including exemplary hardware components. In embodiments, the image correction system 110 may include one or more processor(s) 902 for controlling overall operation of the image correction system 110 and its associated components, including memory(s) 904, network interface(s) 906, and/or input/output devices(s) 908. A data bus 910 may interconnect the processor(s), memory(s), I/O device(s), and/or network interface(s). In some embodiments, the image correction system 110 may represent, be incorporated in, and/or include various devices such as a desktop computer, a computer server, a mobile device, such as a laptop computer, a tablet computer, a smart phone, any other types of mobile computing devices, and the like, and/or any other type of data processing device.

Software may be stored within the memory 904 of the image correction system 110 to provide instructions to the processor(s) to allow the image correction system 110 to perform various actions. For example, the memory may store software used by the image correction system 110, such as an operating system, software for processing data and/or providing data to client devices, and an associated internal database (e.g., image storage 116). The various hardware memory units in the memory may include volatile and nonvolatile, removable and non-removable media implemented in any method or technology for storage of information such as computer-readable instructions, data structures, program modules, or other data. The memory may include one or more physical persistent memory devices and/or one or more non-persistent memory devices. The memory may include, but is not limited to, random access memory (RAM), read only memory (ROM), electronically erasable programmable read only memory (EEPROM), flash memory or other memory technology, optical disk storage, magnetic cassettes, magnetic tape, magnetic disk storage or other magnetic storage devices, or any other medium that may be used to store the desired information and that may be accessed by the processor(s).

The network interface(s) 906 of the image correction system 110 may include one or more transceivers, digital signal processors, and/or additional circuitry and software for communicating via any network, wired or wireless, using any protocol as described herein.

The processor(s) 902 of the image correction system 110 may include a single central processing unit (CPU), which may be a single-core or multi-core processor or may include multiple CPUs. The processor(s) and associated components may allow the image correction system 110 to execute a series of computer-readable instructions to perform some or all of the processes described herein. Although not shown in FIG. 9, various elements within the image correction system 110 may include one or more caches, for example, CPU caches used by the processor(s), page caches used by the operating system, disk caches of a hard drive, and/or database caches used to cache content from a database. For embodiments including a CPU cache, the CPU cache may be used by one or more processors to reduce memory latency and access time. A processor may retrieve data from or write data to the CPU cache rather than reading/writing to memory, which may improve the speed of these operations. In some examples, a database cache may be created in which certain data from a database is cached in a separate smaller database in a memory separate from the database, such as in RAM or on a separate computing device. For instance, in a multi-tiered application, a database cache on an application server may reduce data retrieval and data manipulation time by not needing to communicate over a network with a back-end database server. These types of caches and others may be included in various embodiments and may provide potential advantages in certain implementations of devices, systems, and methods described herein, such as faster response times and less dependence on network conditions when transmitting and receiving data.

Although various components of the image correction system 110 are described separately, functionality of the various components may be combined and/or performed by a single component and/or multiple computing devices in communication.

The various systems, and devices described herein may have similar or different architecture as described with respect to the image correction system 110. Those of skill in the art will appreciate that the functionality of the image correction system 110 as described herein may be spread across multiple data processing devices, for example, to distribute processing load across multiple computers, to segregate transactions based on geographic location, user access level, quality of service (QoS), etc.

One or more aspects discussed herein may be embodied in computer-usable or readable data and/or computer-executable instructions, such as in one or more program modules, executed by one or more computers or other devices as described herein. Generally, program modules include routines, programs, objects, components, data structures, and the like. that perform particular tasks or implement particular abstract data types when executed by a processor in a computer or other device. The modules may be written in a source code programming language that is subsequently compiled for execution or may be written in a scripting language such as (but not limited to) HTML or XML. The computer executable instructions may be stored on a computer readable medium such as a hard disk, optical disk, removable storage media, solid-state memory, RAM, and the like. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like. Particular data structures may be used to more effectively implement one or more aspects discussed herein, and such data structures are contemplated within the scope of computer executable instructions and computer-usable data described herein. Various aspects discussed herein may be embodied as a method, a computing device, a system, and/or a computer program product.

Although the present disclosure has been described using certain specific aspects, many additional modifications and variations would be apparent to those skilled in the art. In particular, any of the various processes described above may be performed in alternative sequences and/or in parallel (on different computing devices) in order to achieve similar results in a manner that is more appropriate to the requirements of a specific application. It is therefore to be understood that the techniques described herein may be practiced otherwise than specifically described. Thus, embodiments should be considered in all respects as illustrative and not restrictive. Accordingly, the scope of the invention should be determined not by the embodiments illustrated, but by the appended claims and their equivalents.

What is claimed is:

1. A computer-implemented method for processing fundus imaging data to generate a calibrated eye measurement, the method comprising:
receiving fundus imaging data comprising a plurality of images of a fundus of an eye, wherein the fundus imaging data is captured using a multispectral camera configured to capture images corresponding to different spectral bands;
receiving reference imaging data comprising a plurality of images of a reference model of an eye, wherein the reference model is a physical artificial eye, wherein the reference imaging data is captured using the multispectral camera;
adjusting the fundus imaging data to match the reference imaging data or the reference imaging data to match the fundus imaging data to yield post-adjusting fundus imaging data and post-adjusting reference imaging data by performing one or more of:
compensating for a manufacturing imperfection in the reference model;
compensating for a field of view difference between the fundus imaging data and the reference imaging data;
compensating for a diffusivity difference between the human eye and the reference model; or
compensating for a spectral difference in the media between the reference model and the human eye; and
generating a calibrated eye measurement based on processing the post-adjusting fundus imaging data and the post-adjusting reference imaging data.

2. The method of claim 1, wherein the adjusting comprises compensating for the field of view difference between the fundus imaging data and the reference imaging data by:
detecting a size difference and a position difference between the fundus imaging data and the reference imaging data; and
transforming one of the fundus imaging data or the reference imaging data to remove the size difference and the position difference.

3. The method of claim 2, further comprising detecting a first field of view of the fundus imaging data and a second field of view of the reference imaging data by detecting which pixels of the corresponding imaging data are associated with an intensity value that is greater than a threshold level.

4. The method of claim 1, wherein the adjusting comprises compensating for the diffusivity difference between the human eye and the reference model by:
quantifying the diffusivity difference between the human eye and the reference model on a wavelength-by-wavelength basis; and
for each wavelength of a set of one or more wavelengths, correcting the diffusivity difference between a fundus image associated with the wavelength and a reference image associated with the same wavelength by blurring whichever image is associated with a lesser diffusivity to match the corresponding image.

5. The method of claim 4, wherein quantifying the diffusivity difference comprises using predetermined wavelength-specific diffusivity factors.

6. The method of claim 4, wherein the blurring comprises using a gaussian filter with a window size that is dependent on the corresponding wavelength.

7. The method of claim 1, wherein the adjusting comprises compensating for the manufacturing imperfection in the reference model by:
for each wavelength of a of a set of one or more wavelengths captured by the multispectral camera, combining a plurality of images of the reference model taken from different orientations to generate a combined image for the corresponding wavelength;

8. The method of claim 7, wherein the adjusting further comprises, for each wavelength of the set of one or more wavelengths, applying at least one edge-preserving filter to the corresponding averaged image, wherein the at least one edge-preserving filter comprises a median filter and a non-local means denoising algorithm.

9. The method of claim 1, wherein the adjusting comprises compensating for the spectral difference in the media between the reference model and the human eye by:
for each wavelength of a set of one or more wavelengths captured by the multispectral camera, applying a corresponding correction coefficient for the human eye.

10. The method of claim 1, wherein the fundus imaging data is captured by the multispectral camera using a rolling shutter acquisition, wherein the method further comprises:
determining, for one or more horizontal rows of the fundus imaging data, a central wavelength; and
removing a vertical spectral gradient in the fundus imaging data by interpolating between different images of the fundus imaging data for each of the one or more horizontal rows based on the corresponding central wavelength.

11. The method of claim 1, wherein adjusting the fundus imaging data to match the reference imaging data or the reference imaging data to match the fundus imaging data further comprises compensating for temporal light fluctuations in the fundus imaging data and the reference imaging data by adjusting based on an illumination power measurement to perform a power correction.

12. The method of claim 11, wherein compensating for temporal light fluctuations in the fundus imaging data and the reference imaging data further comprises using dark image subtraction, wherein the dark image subtraction comprises subtracting at least one dark image captured using the multispectral camera; and

13. The method of claim 1, wherein adjusting the fundus imaging data to match the reference imaging data or the reference imaging data to match the fundus imaging data further comprises compensating for parasitic reflections of optics of the multispectral camera in the fundus imaging data and the reference imaging data using baseline imaging data, wherein the baseline imaging data is captured using the multispectral camera, wherein the baseline imaging data comprises a plurality of images of a light trap.

14. The method of claim 1, further comprising receiving a selection of a subset of the fundus imaging data, wherein the subset specifies one or more of a subset of wavelengths or a subset of pixels, wherein the adjusting and the generating are limited to the subset of the fundus imaging data.

15. A computer-implemented system, the system comprising a processor and a memory storing a plurality of executable instructions which, when executed by the processor, cause the system to perform the method of:
receiving fundus imaging data comprising a plurality of images of a fundus of an eye, wherein the fundus imaging data is captured using a multispectral camera configured to capture images corresponding to different spectral bands;
receiving reference imaging data comprising a plurality of images of a reference model of an eye, wherein the reference model is a physical artificial eye, wherein the reference imaging data is captured using the multispectral camera;
adjusting the fundus imaging data to match the reference imaging data or the reference imaging data to match the fundus imaging data to yield post-adjusting fundus imaging data and post-adjusting reference imaging data by performing one or more of:
compensating for a manufacturing imperfection in the reference model;
compensating for a field of view difference between the fundus imaging data and the reference imaging data;
compensating for a diffusivity difference between the human eye and the reference model; or
compensating for a spectral difference in the media between the reference model and the human eye; and
generating a calibrated eye measurement based on processing the post-adjusting fundus imaging data and the post-adjusting reference imaging data.

16. A non-transitory computer readable medium comprising control logic which, upon execution by a processor, causes execution of the method of:
receiving fundus imaging data comprising a plurality of images of a fundus of an eye, wherein the fundus imaging data is captured using a multispectral camera configured to capture images corresponding to different spectral bands;
receiving reference imaging data comprising a plurality of images of a reference model of an eye, wherein the reference model is a physical artificial eye, wherein the reference imaging data is captured using the multispectral camera;
adjusting the fundus imaging data to match the reference imaging data or the reference imaging data to match the fundus imaging data to yield post-adjusting fundus imaging data and post-adjusting reference imaging data by performing one or more of:
compensating for a manufacturing imperfection in the reference model;
compensating for a field of view difference between the fundus imaging data and the reference imaging data;
compensating for a diffusivity difference between the human eye and the reference model; or
compensating for a spectral difference in the media between the reference model and the human eye; and
generating a calibrated eye measurement based on processing the post-adjusting fundus imaging data and the post-adjusting reference imaging data.
